(19) United States
(12) Patent Application Publication
(10) Pub. No.: US 2017/0049842 A1
(43) Pub. Date: Feb. 23, 2017

(54) FOOD ADDITIVE FOR PRODUCING FOOD FOR PREVENTING CRANIAL NERVE DISEASE AND/OR IMPROVING BRAIN FUNCTION

(71) Applicant: FUJI OIL HOLDINGS INC., Osaka (JP)

(72) Inventors: Shigeki FURUYA, Fukuoka-shi, Fukuoka (JP); Toshiro MATSUI, Fukuoka-shi, Fukuoka (JP); Mitsuru TANAKA, Fukuoka-shi, Fukuoka (JP); Motohiro MAEBUCHI, Moriya-shi, Ibaraki (JP); Toshihiro NAKAMORI, Nara-shi, Nara (JP); Hitoshi FURUTA, Moriya-shi, Ibaraki (JP)

(73) Assignee: FUJI OIL HOLDINGS INC., Osaka (JP)

(21) Appl. No.: 15/306,802

(22) PCT Filed: Apr. 28, 2015
(86) PCT No.: PCT/JP2015/062778

§ 371 Date: Oct. 26, 2016

(30) Foreign Application Priority Data
Apr. 28, 2014 (JP) 2014-092431

(51) Int. Cl. A61K 38/05; A23L 33/17

(52) U.S. Cl. CPC A61K 38/05; A23L 33/17; A23V 2002/00

(57) ABSTRACT

Provided is a raw material for a food additive which promotes the intracerebral release of monoamines such as dopamine and noradrenaline, and imparts a function to prevent cranial nerve disease and a function to improve brain function to foods, by being added to said foods. This method involves using as a food additive an oligopeptide mixture containing dipeptides or tripeptides having tyrosine or phenylalanine as constituent amino acids, in order to produce foods to prevent cranial nerve disease or foods to improve brain function.

### TECHNICAL FIELD

The present invention relates to a food additive for producing food products which are used to prevent cranial nerve diseases or to improve brain function.

### BACKGROUND ART

With an increase in the aging population, the number of patients of senile dementia including Alzheimer's disease, is increasing. According to the Ministry of Health, Labour and Welfare, the elderly with dementia is estimated to increase to 4.1 million people in 2020 from 2.8 million people in 2010.

In addition, the number of patients which are having troubles of the brain, such as depression according to various stresses such as work environment, family circumstances, and human relations, is increasing year by year. Recent studies demonstrate that food components affect the function of the brain. Thus, a food component having an effect related to brain function improvement, antidepressant, and antidementia, has attracted attention.

Methods for improving brain function have been studied from the past, such as, a method for improving a metabolism of brain energy to activate the function of cells (for example, an increase in brain glucose), a method for improving cerebral circulation which provides enough nutrients and oxygen which are necessary for brain cells by improving blood circulation (for example, an increase in cerebral blood flow), a method for activating a nerve transmission which is performed in the synaptic cleft through the neurotransmitter (supply of the precursor of the neurotransmitter (for example, supply of choline or acetyl CoA)), an inhibition of a conversion of released neurotransmitter (for example, acetylcholinesterase inhibition), an increase of neurotransmitter release (for example, increased release of acetylcholine or glutamate), an activation of neurotransmitter receptors, or a protection of the nerve cell membrane (for example, anti-oxidation, supply of membrane components, or prevention of arteriosclerosis).

Dopamine and noradrenaline are a neurotransmitter which presents in the central nervous system, and are collectively referred to as monoamine neurotransmitter along with adrenaline, serotonin, and histamine. In addition, dopamine is also a precursor of noradrenaline. Dopamine relates to motor control, hormonal regulation, free of emotion, motivation, and learning. In addition, it has been suggested that cognitive functions such as planning and working memory are involved dopamine (Non-Patent Documents 1 and 2). Meanwhile, it is known that noradrenaline relates to cognitive functions such as maintenance of wakefulness, modulation of sensory input, formation of long-term memory, and attention (Non-Patent Documents 3 and 4). It is also a working target portion of antidepressants.

Thus, development of a food material, which prevents or improves symptoms or diseases caused by decreased brain functions by increasing brain level of monoamines including dopamine and noradrenaline, and which has high-safety, is strongly desired.

### PRIOR ART DOCUMENTS

### Non-Patent Documents

Non-Patent Document 1: Nieoullon A. Dopamine and the regulation of cognition and attention. Prog Neurobiol. 2002; 67: 53-83.

Non-Patent Document 2: Cools R and Robbins T W. Chemistry of the adaptive mind. Phil Trans R Soc Lond A. 2004; 362: 2871-88.

Non-Patent Document 3: Foote S L, Freedman R, Oliver A P. Effects of putative neurotransmitters on neuronal activity in monkey auditory cortex. Brain Res. 1975; 86: 229-42.

Non-Patent Document 4: McGaugh J L and Roozendaal B. Drug enhancement of memory consolidation: historical perspective and neurobiological implications.

Psychopharmacology (Berl). 2009; 202: 3-14. doi:10.1007/s00213-008-1285-6.

### SUMMARY OF INVENTION

### Problems to be Solved by Invention

An object of the present invention is to provide a material for food additive which enables to add a function of preventing cranial nerve diseases or a function of improving a brain function by promoting brain release of monoamines, including dopamine and noradrenaline, to food when the food additive is added to the food.

### Means for Solving the Problems

The present inventors have extensively studied high-safety materials of food additive, which increase brain monoamine levels efficiently. As a result, they have found that a dipeptide or tripeptide which has tyrosine as one of the constituent amino acids, or oligopeptide mixture containing several amount of the peptide increases brain monoamine levels efficiently. The present invention has been completed based on the findings.

That is, the present invention includes:

(1) A method for using an oligopeptide mixture as a food additive, the oligopeptide mixture containing dipeptide or tripeptide having tyrosine or phenylalanine as a constituent amino acid, for preparing a food for preventing a cranial nerve disease or a food for improving a brain function;

(2) The method of (1), where a ratio of tyrosine and phenylalanine to total amino acids in the oligopeptide mixture is 5% by weight or more;

(3) The method of (1), where an amount of peptide having less than 500 of molecular weight is 50% by weight or more with respect to a total amount of peptide and free amino acid;

(4) The method of (1) or (2), where the dipeptide or tripeptide having tyrosine or phenylalanine as a constituent amino acid acts as an active component of promoting brain release of monoamines;

(5) The method of (4), where the dipeptide as an effective component is one or two or more dipeptides selected from the group consisting of Ser-Tyr, Ile-Tyr, and Tyr-Pro; and

(6) A method of using a dipeptide or tripeptide having tyrosine or phenylalanine as a constituent amino acid, for producing a food which is used to prevent cranial nerve diseases or to improve brain function, including adding the dipeptide or tripeptide, which acts as an active component, to the food.

### Effect of the Invention

By ingesting a specific dipeptide or tripeptide of the present invention as an active component, a brain release of monoamines such as dopamine and noradrenaline may be promoted. This may be useful to prevent various cranial nerve disorders and to improve cerebral functions.

### Mode for Carrying Out the Invention

Embodiments of the present invention will be described in detail in the following.

### (Oligopeptide Mixture)

An oligopeptide mixture is not a peptide having only a specific amino acid sequence, but a mixture of peptides having various amino acid sequence and molecular weight.

In one embodiment, the oligopeptide mixture of the present invention used as a food additive may be a protein acid hydrolysate which is obtained by hydrolyzing protein material with an acid or a protein enzyme degradation product which is obtained by degrading protein with a proteolytic enzyme (protease). Otherwise, it may also be prepared in a conventional manner, such as chemical synthesis and enzymatic methods.

An aspect of obtaining the oligopeptide mixture by the enzymatic degradation will be described in the following. Various protein materials, which are obtained by extracting, concentrating or isolating protein from animal-derived or plant-derived natural materials, may be used as a protein material. Preferred protein content of the protein material is 50% by weight or more, preferably 70% by weight or more, more preferably 80% by weight or more, further preferably 90% by weight or more, on the dry weight basis.

Examples of an origin of the animal-derived protein material include milk, egg, livestock, fish and seafood, and microorganism. Examples of an origin of the plant-derived protein material include bean such as soybean and pea, and cereal such as rice, wheat, barley and corn.

Among them, an origin containing a large amount of aromatic amino acid, tyrosine residue or phenylalanine residue, which is raw material of tyrosine, in the amino acids of the protein is preferable. Examples of such an origin include soybean, milk, livestock, fish and seafood and egg. Preferably, it is soybean. In the case of soybean, soymilk, which may be full-fat or defatted, concentrated soybean protein, isolated soybean protein, or fractionated soybean protein may be used. Especially, if an intake of large amount of oligopeptide is desired with small amount of intake, use of isolated soybean protein or fractionated soybean protein, which have 80% by weight or more of protein content on the dry basis, is preferable.

A preferred degree of the enzyme degradation of the protein material with a proteolytic enzyme (protease) is that all molecules are not completely degraded to free amino acids. In addition, higher degradation rate is preferable. Especially, a content of peptide fraction having less than 500 of molecular weight is preferably 50% by weight or more, preferably 60% by weight or more with respect to a total amount of peptides and free amino acids.

The peptide having less than 500 of molecular weight is substantially composed of dipeptide and tripeptide in which 2 or 3 molecules of amino acids are bound.

When the molecular weight of the oligopeptide mixture is too large, advantage of absorption rate is reduced and the effect of promoting release of monoamines might be diminished.

The content of peptide having less than 500 of molecular weight is calculated by determining the rate of peptides having less than 500 of molecular weight and free amino acids in the oligopeptide mixture with a gel filtration chromatography for peptide, then subtracting the free amino acid content, which is calculated by an amino acid analysis, in the protein hydrolysate.

Preferably, the oligopeptide mixture identified as described above has lower content of peptide other than the peptide having less than 500 of molecular weight and lower content of free amino acids. That is, the content of free amino acids in the oligopeptide mixture is preferably 12% by weight or less, preferably 5% by weight or less with respect to a total amount of peptide and free amino acids. This is because high intake of free amino acids may cause problems when the content of free amino acids is too high.

Further, since it is desirable that peptide in the oligopeptide mixture is lower molecular weight, the rate of fraction having 500 or more of molecular weight to peptide and free amino acids in the oligopeptide mixture is preferably 40% by weight or less, more preferably 38% by weight or less, further preferably 35% by weight or less.

Protease used in the enzyme degradation in order to obtain the oligopeptide mixture may be selected from any proteases, such as “metalloprotease”, “acid protease”, “thiol protease” and “serine protease”, in the classification of proteases, preferably selected from proteases classified into “metal protease”, “thiol protease” or “serine protease”, regardless of animal-, plant- or microorganism-origin.

Especially, a method of degrading with enzymes belonging to two or three or more different classifications in series in combination sequentially or simultaneously enables to increase the ratio of peptide having less than 500 of molecular weight. Therefore, such a method is efficient and preferable.

Further, it is preferred to use enzyme having less exoprotease activity in order to reduce the content of free amino acids.

This classification of protease is normally carried out in the field of enzyme science, i.e. a method of classification according to the kind of amino acid in the active center.

As typical examples of each enzyme, “metalloprotease” includes Bacillus-derived neutral protease, Streptomyces-derived neutral protease, Aspergillus-derived neutral protease, and “Thermoase”; “acid protease” includes pepsin, Aspergillus-derived acid protease, and “Sumizyme FP”; “thiol protease” includes bromelain, and papain; and “serine protease” includes trypsin, chymotrypsin, subtilisin, Streptomyces-derived alkaline protease, “Alcalase”, and “Bioprase”.

The classification of other enzymes may be confirmed by the working pH and reactivity with inhibitors.

Enzymes having different active center enable to obtain an enzymatic degradation product effectively because the active site to a substrate is very different between such enzymes and “uncut portions” are reduced.

In addition, an enzyme degradation product may be produced effectively by using enzymes derived from different-origins (source organisms) in combination.

Enzymes belonging to the same classification, but derived from different-origins act to different active site of a substrate protein. As a result, it is possible to increase a ratio of peptides having less than 500 of molecular weight.

Reaction pH and reaction temperature of the protease treatment may be set to match the characteristics of the protease used. Usually, a reaction may be carried out at near the optimum pH and near the optimum temperature.

Generally, the reaction temperature is 20 to 80° C., preferably 40 to 60° C. After the reaction, the residual enzyme activity is inactivated by heating to a sufficient temperature (about 60 to 170° C.) to deactivate the enzyme.

The reaction solution after the protease treatment may be used directly or after concentrated. Typically, the solution is used in powder form after sterilization, splay-drying, or freeze-drying.

Heat sterilization is preferred as a sterilization. And the heat temperature is preferably 110 to 170° C., more preferably 130 to 170° C., and the heating time is preferably 3 to 20 seconds. In addition, the reaction solution may be adjusted to any pH.

The insoluble matter (precipitate or suspension) generated in the protease treatment and pH adjustment may be removed by centrifugation or filtration. The removal of the insoluble matter is preferable because titer of the active ingredients in the oligopeptide mixture may be improved. In addition, it may be further purified by activated carbon or adsorbent resin.

### (Dipeptide or Tripeptide Having Tyrosine or Phenylalanine as a Constituent Amino Acid: ARPs)

It is important that the oligopeptide mixture used as a food additive in the present invention contains a “dipeptide or a tripeptide having tyrosine or phenylalanine as a constituent amino acid” [hereinafter, it is abbreviated as “ARPs” (Aromatic Peptides). That is, the present invention basically relates to an action of the ARPs as an active component for promoting the release (secretion and turnover) of monoamines from brain nerve cells. Incidentally, phenylalanine is a precursor of tyrosine, and therefore tyrosine is produced from phenylalanine in the body. Thus, an ingestion of phenylalanine may be substantially same as that of tyrosine.

The ARPs contain one or two tyrosine or phenylalanine residues in dipeptide, or 1 to 3 tyrosine or phenylalanine residues in tripeptide.

Tyrosine residue or phenylalanine residue may be present in N-terminal or C-terminal of ARPs, or in the middle of the amino acid sequence in the case of tripeptide. In addition, peptide transporters, which are independent from amino acid transporters, are in the gastrointestinal tract. And, it is known that both dipeptides and tripeptides are transported into cells as peptide form. Thus, an ingestion of tripeptide may be substantially same as that of dipeptide (Adibi S A, The oligopeptide transporter (Pept-1) in human intestine: biology and function, Gastroenterology, 1997; 113: 332-340.).

In addition, ARPs contained in the oligopeptide mixture may be a mixture of those having two or more amino acid sequence including tyrosine or phenylalanine as well as those of single amino acid sequence including tyrosine or phenylalanine.

Among the ARPs used in the present invention, those having higher permeability coefficient (Pₐₚₚ) of intestinal membrane model cells are particularly preferable. More preferably, the permeability coefficient (Pₐₚₚ) is preferably at 15×10⁻⁸ cm/sec or more, more preferably 40×10⁻⁸ cm/sec or more, and further preferably at 65×10⁻⁶ cm/sec or more, as measured by the method described in Examples. The permeability coefficient is used as an index of ease of pass of ARPs in peptide transporters present in an intestinal tract.

As ARPs satisfying such an index, for example, dipeptide selected from the group consisting of Ile-Tyr, Tyr-Pro, Ser-Tyr, Tyr-Leu and Tyr-Ser, especially, dipeptide selected from the group consisting of Ile-Tyr, Tyr-Pro and Ser-Tyr, is preferable.

It is considered that an amount of ARPs may be higher when a ratio of tyrosine and phenylalanine to total amino acids of the oligopeptide mixture is higher. Thus, the ratio is preferably high, more specifically, from 5% by weight to 80% by weight.

Although it is not an essential, when the content of ARPs in the oligopeptide mixture is desired to be further enhanced, enzyme degradation product of protein may be further concentrated or purified after the enzyme degradation of protein material with a proteolytic enzyme.

In addition, oligopeptide mixture containing ARPs used in the present invention may be prepared by enzyme process using plastein reaction or amino acid ligase, or by chemical synthesis. However, it is preferable to concentrate or purify the protein hydrolysate with considering the economy, efficiency and use as a food material.

Concentration may be carried out by adsorbing fraction containing a large amount of ARPs in an oligopeptide mixture using an adsorbent or the like.

And, purification may be carried out by adding polar organic solvent such as ethanol a solution of the oligopeptide mixture, and then removing precipitate and recovering the soluble fraction to obtain a fraction rich in ARPs (International Publication No. WO 2008/123033).

### (Physiology of ARPs)

The present inventors have found that a metabolic turnover rate of monoamines such as dopamine and noradrenaline becomes significantly higher in the cerebral cortex and hippocampus of the brain of mouse compared to the control by administering Ile-Tyr, Tyr-Pro, or Ser-Tyr, which shows relatively high permeation efficiency in mesenteric model cell, to the mouse.

Although not to the extent of the above dipeptides, since Tyr-Leu and Tyr-Ser shows relatively higher permeability coefficient than the other ARPs, it is supported that high metabolic turnover rate is provided by administering these dipeptides to mouse.

The present inventors have found from the proven results that a promoting effect of brain release of monoamines such as dopamine and noradrenaline is obtained by ingesting ARPs, more preferably oligopeptide mixture containing ARPs showing relatively high permeation efficiency in the mesenteric model cell.

### (Use of ARPs-containing Oligopeptide Mixture as a Food Additive)

From the above physiology, the oligopeptide mixture containing ARPs as active component may be used as a food additive for imparting the above physiology in order to produce food products for prevention of cranial nerve diseases or improvement of brain function.

The “food additive” in the present invention is intended to mean a raw material for imparting particular functions to food by adding. It is not limited to a food additive which is regulated by law in various countries, but means broader concept.

ARPs-containing oligopeptide mixture used as a food additive of the present invention may be utilized for food in various forms. For example, it may be used as a raw material which is added to products such as beverage, tablet, food bar, meat product, dessert, confectionery and food supplement.

These products may clearly show the effect of prevention of cranial nerve diseases or improvement of brain function in their packaging or advertising media. Product without showing such an effect, but the seller of the product intends or expects to impart such an effect by adding the food additive of the present invention, may also be included in the products containing the food additive of the present invention.

### (Cranial Nerve Disease)

Examples of cranial nerve disease include higher brain function disorders such as memory impairment, attention disorders, executive function impairment, and social behavior disorders; and symptoms relevant to these disorders and pathologically, for example, cerebral infarction, head trauma, brain vascular dementia, Alzheimer's type dementia, Parkinson's disease, schizophrenia, depression, and anxiety.

### (Improvement in Brain Function)

Specifically, the effect of improvement in brain function includes memory improvement, improvement in learning ability, improvement in attentional capacity, stress tolerance, anti-depressant effect, anti-anxiety effect, concentration improvement, and improvement in sleep quality.

### (Measuring Method of Free Amino Acid and Peptide Content)

The molecular weight distribution of an oligopeptide mixture is determined by HPLC method using the following gel filtration column.

HPLC system using a gel filtration column for peptide is assembled, and then a known peptide as a molecular weight marker is charged to determine a calibration curve at the relationship between retention time and molecular weight. β-Asp-Arg-Val-Tyr-Ile-His-Pro-Phe (molecular weight: 1046) of [β-Asp]-Angiotensin II as octapeptide, Val-Tyr-Ile-His-Pro-Phe (molecular weight: 775) of Angiotensin IV as hexapeptide, and Tyr-Gly-Gly-Phe-Leu (molecular weight: 555) of Leu-Enkephalin as penta peptide, Glu-Glu-Glu (molecular weight: 405) as a tripeptide, and Pro (molecular weight: 115) as a free amino acid are used as a molecular weight marker.

An oligopeptide mixture (1%) is centrifuged at 10,000 rpm for 10 minutes, the obtained supernatant is diluted 2-fold with gel filtration solvent, and then 5 μl of it is applied into HPLC.

A ratio (%) of free amino acids and peptide fractions having less than 500 of molecular weight in a protein hydrolysate is determined from the ratio of area in the range of less than 500 of molecular weight (a time range) to the chart area of the entire absorbance, (using column: Superdex Peptide 7.5/300 GL (manufactured by GE Healthcare Japan Co., Ltd.), solvent: 1% SDS/10 mM phosphate buffer, pH: 8.0, column temperature: 25° C., flow rate: 0.25 ml/min, detection wavelength: 220 nm).

A ratio (%) of the peptide fractions having 500 or more of molecular weight in the protein hydrolysate is determined from the ratio of area in the range of 500 or more of molecular weight to the chart area of the entire absorbance as described in the above.

Then, the measurement of the free amino acid content in the protein hydrolysate is determined by amino acid analysis. The protein hydrolysate (4 mg/ml) is added to equal amount of 3% sulfosalicylic acid, and then shaken for 15 minutes at room temperature. The mixture is centrifuged at 10,000 rpm for 10 minutes. The obtained supernatant is filtered through a 0.45 pm filter, and then applied to amino acid analyzer “JLC500V” (manufactured by JEOL Ltd.) to determine free amino acid.

Free amino acid content of the protein hydrolysate is calculated as a ratio of the protein content which is obtained by Kjeldahl method.

A “content of peptides having less than 500 of molecular weight” in the protein hydrolysate is a value obtained by subtracting the “free amino acid content” from the “ratio of free amino acids and peptide fraction having less than 500 of molecular weight” obtained from the above.

### EXAMPLES

The present invention will be described in more detail below by way of examples of the present invention.

To obtain ARPs contained in a soybean-derived oligopeptide mixture, dipeptides including tyrosine and amino acid prior to or after the tyrosine from 7S globulin and 11S globulin in the sequence were listed, and the following 8 dipeptides (peptides A-H) showing high appearance frequency were selected as ARPs as shown in the following table 1. These peptides were chemically synthesized for testing.

TABLE 1
Selected 8 APRs

| | |
|---|---|
| (A) | Ser-Tyr |
| (B) | Tyr-Leu |
| (C) | Tyr-Arg |
| (D) | Tyr-Ser |
| (E) | Tyr-Pro |
| (F) | Tyr-Asn |
| (G) | Phe-Tyr |
| (H) | Ile-Tyr |

The measurement of permeability coefficient, which was an index of absorbability in the intestinal tract, of ARPs was performed as follows.

Caco-2 cells were seeded in Cell culture insert at 4.0×10⁵ cells/mL, and cultured for 3 days in an intestinal epithelial differentiation promoting medium. Caco-2 cell monolayers were cut and set to Ussing Chamber. Hanks' Balanced Salt Solution (HBSS) (apical membrane side: pH 6.0, basolateral membrane side: pH 7.4) was added in each Chamber. After 15 minutes preliminary heat retention (37° C., 95% O₂/5% CO₂ mixture gas), each ARPs aqueous solution (10 mM) was added to apical membrane side. Samples were recovered from the basolateral membrane side every 15 minutes (total 60 minutes). The recovered sample was subjected to ESI-TOF-MS (Electro Spray Ionization-Time of Flight-Mass Spectrometry) analysis to determine permeated peptide amount. Permeability coefficient (Pₐₚₚ) was calculated according to the following formula. In addition, the analytical conditions of ESI-TOF-MS were shown in the following Table 2.

### [Mathematical Formula 1]

Pₐₚₚ(cm/sec)=V/AC₀×dC/dt

V: HBSS amount (ml)

A: Membrane area (cm²)

C₀: concentration of added peptide (mmol/L)

dC/dt: Permeated amount of peptide per time (mmol/L·sec)

TABLE 2
Analytical conditions of ESI-TOF-MS

| | | |
|---|---|---|
| Column | Peptides | “Cosmosil 5C18-MS-II” |
| used | A-G | (φ2.0 mm × 150 mm, |
| | | manufactured by |
| | | NACALAI TESQUE, INC.) |
| | Peptide | “Atlantis T3” |
| | H | (φ2.1 mm × 100 mm, |
| | | manufactured by |
| | | Chromato Research, Inc.) |

| Eluent | Gradient from 0 to 100% by volume methanol | |
|---|---|---|
| | (including 0.1% folic acid) | |
| Column | 40° C. | |
| temperature | | |
| Flow rate | 0.2 | mL/min |
| Injection | 20 | μL |

| volume | | | | |
|---|---|---|---|---|
| MS | Mode | Positive-low, Expert mode | | |
| condition | Nebulizer | 1.6 | Bar | |
| | Dry Gas | 8.0 | L/min | |
| | Mass range | 50-1000 | | |
| | Hexapole RF | Peptide C | 120 | Vpp |
| | | Other peptides | 100 | Vpp |
| | Capillary Exit | Peptide C | 100 | V |
| | | Other peptides | 70 | V |

Concentration of peptide, which transitioned to the basolateral membrane side in each time, was calculated based on the area from reference standard. In addition, it was confirmed that they were not degraded to amino acids.

Permeated peptide amount per time was calculated and permeability coefficient (Pₐₚₚ) was calculated according to the above formula. The result of permeation test is shown in table 3.

TABLE 3
Permeability coefficient of tyrosine-containing dipeptide
derived from soybean oligopeptide mixture

| | Amino acid | Appearance | Pₐₚₚ × 10⁻⁸ |
|---|---|---|---|
| ARPs | sequence | frequency* | (cm/sec) |
| A | Ser-Tyr | 16 | 72.0 ± 27.2 |
| B | Tyr-Leu | 8 | 59.6 ± 19.8 |
| C | Tyr-Arg | 8 | 5.0 ± 1.1 |
| D | Tyr-Ser | 5 | 24.8 ± 8.8 |
| E | Tyr-Pro | 5 | 88.5 ± 21.5 |
| F | Tyr-Asn | 5 | 4.4 ± 0.9 |
| G | Phe-Tyr | 5 | 0.6 ± 0.01 |
| H | Ile-Tyr | 5 | 291.9 ± 26.5 |

The test was carried out triply per group.
Each value is shown as average standard deviation.
*Appearance frequency was counted from the sequence of 7S or 11S.

From the results in Table 3, three ARPs of which permeability coefficient was relatively high, more than 65 cm/sec, Peptides A (Ser-Tyr), E (Tyr-Pro), and H (Ile-Tyr) were chemically synthesized and subjected to the animal test. Mouse (C57BL/6NCrlCrlj) was purchased and habituated for 24 hours (using the 10-11 weeks of age).

Each 50 mM ARPs aqueous solution or water (control), 0.6 ml, was forcibly administered with a sonde (0.6 ml/30 g-body weight).

Mouse was dissected after 30 minutes and 60 minutes post-dose, and then the cerebral cortex and hippocampus, which were brain tissues, were taken as samples. Monoamine concentration of each site was measured by using “HTEC-500” (manufactured by Eicom Corporation) as HPLC-ECD (high performance liquid chromatography with an electrochemical detector) system, and then turnover rates of norepinephrine and dopamine were calculated. Turnover rate was calculated according to the following formula. The results were shown in tables 4 and 5.

[Mathematical Formula 2]

Metabolic turnover rate of noradrenaline=MHPG/NE

MHPG: Noradrenaline metabolite concentration (ng/g wet-tissue)

NE: Noradrenaline concentration (ng/g wet-tissue)

Metabolic turnover rate of dopamine=(HVA+DOPAC)/DA

HVA: Dopamine metabolite concentration (ng/g wet-tissue)

DOPAC: Dopamine metabolite concentration (ng/g wet-tissue)

DA: Dopamine concentration (ng/g wet-tissue)

TABLE 4
Comparison of metabolic turnover rate in cerebral cortex compared to control.

| | (A) Ser-Tyr/Control | | | | (H) Ile-Tyr/Control | | | |
|---|---|---|---|---|---|---|---|---|
| Metabolic | 30 min | | 60 min | | 30 min | | 60 min | |
| turnover | Ratio | | Ratio | | Ratio | | Ratio | |
| rate | (times) | t-test | (times) | t-test | (times) | t-test | (times) | t-test |
| Noradrenaline | 2.67 | <0.001 | 3.13 | <0.001 | 1.99 | <0.001 | 1.53 | <0.01 |
| Dopamine | 0.94 | N.S. | 0.98 | N.S. | 0.93 | N.S. | 1.03 | N.S. |

| | (E) Tyr-Pro/Control | | | |
|---|---|---|---|---|
| | 30 min | | 60 min | |
| | Ratio (times) | t-test | Ratio (times) | t-test |
| Noradrenaline | 2.20 | <0.001 | 1.74 | N.S. |
| Dopamine | 0.92 | N.S. | 1.01 | N.S. |

*Ratio: Peptide metabolic turnover rate/Control metabolic turnover rate (%), n = 5-8

TABLE 5
Comparison of metabolic turnover rate in hippocampus compared to control.

| | (A) Ser-Tyr/Control | | | | (H) Ile-Tyr/Control | | | |
|---|---|---|---|---|---|---|---|---|
| | 30 min | | 60 min | | 30 min | | 60 min | |
| | Ratio | | Ratio | | Ratio | | Ratio | |
| | (times) | t-test | (times) | t-test | (times) | t-test | (times) | t-test |
| Noradrenaline | 2.23 | <0.001 | 2.47 | <0.001 | 1.74 | <0.001 | 1.34 | <0.05 |
| Dopamine | 0.98 | N.S. | 1.17 | N.S. | 1.08 | N.S. | 1.27 | <0.05 |

| | (E) Tyr-Pro/Control | | | |
|---|---|---|---|---|
| | 30 min | | 60 min | |
| | Ratio (times) | t-test | Ratio (times) | t-test |
| Noradrenaline | 1.76 | <0.001 | 1.45 | N.S. |
| Dopamine | 0.95 | N.S. | 1.04 | N.S. |

*Ratio: Peptide metabolic turnover rate/Control metabolic turnover rate (%), n = 5-8

As shown in tables 4 and 5, the turnover rate of noradrenaline in the cerebral cortex and hippocampus was significantly higher in the administration of each ARPs relative to the control. And, that of dopamine was higher in each peptide in the hippocampus.

That is, it was shown that secretion and turnover of monoamines such as dopamine and noradrenaline (i.e. brain release) was promoted by ingestion of ARPs contained in an oligopeptide mixture.

1-6. (canceled)

7. A method for treating or preventing a cranial nerve disease, which comprises administering a dipeptide or a tripeptide having tyrosine or phenylalanine as a constituent amino acid.

8. The method according to claim 7, wherein a ratio of tyrosine and phenylalanine to total amino acids in the dipeptide or the tripeptide is 5% by weight or more.

9. The method according to claim 7, wherein the dipeptide or the tripeptide is comprised in a food additive.

10. The method according to claim 7, wherein the dipeptide is one or two or more of dipeptides selected from a group consisting of Ser-Tyr, Ile-Tyr and Tyr-Pro.

11. The method according to claim 7, wherein the dipeptide is Ser-Tyr.

12. A method for enhancing an intracerebral release of monoamine, which comprises administering a dipeptide or a tripeptide having tyrosine or phenylalanine as a constituent amino acid.

13. The method according to claim 12, wherein a ratio of tyrosine and phenylalanine to total amino acids in the dipeptide or the tripeptide is 5% by weight or more.

14. The method according to claim 12, wherein the dipeptide or the tripeptide is comprised in a food additive.

15. The method according to claim 12, wherein the dipeptide is one or two or more of dipeptides selected from a group consisting of Ser-Tyr, Ile-Tyr and Tyr-Pro.

16. The method according to claim 12, wherein the dipeptide is Ser-Tyr.
